(19) United States
(12) Patent Application Publication
(10) Pub. No.: US 2003/0072455 A1
(43) Pub. Date: Apr. 17, 2003

(54) Method and system for generating audio streams during a hearing test

(76) Inventors: Benny B. Johansen, Sunnyvale, CA (US); Ephram Cohen, San Francisco, CA (US); Sunil Puria, Mountain View, CA (US)

Correspondence Address: HAYNES BEFFEL & WOLFELD LLP, P O BOX 366, HALF MOON BAY, CA, 94019, US

(21) Appl. No.: 09/975,047

(22) Filed: Oct. 11, 2001

(51) Int. Cl. H04R 29/00; A61B 5/00

(52) U.S. Cl. 381/60; 600/559

(57) ABSTRACT

A method of testing the hearing of a user utilizing a computer system. The computer system includes a computer and a speaker. The computer can output an electrical signal to the speaker. The speaker can convert the electrical signal into a stimulus. The method includes: downloading a computer program from a server to the computer; executing the computer program on the computer, the execution of the computer program generating an audio stream; based upon the audio stream, generating a stimulus; and receiving an input from the user that indicates that the user heard the stimulus.

This patent application discloses subject matter that is related to the subject matter disclosed in U.S. patent application Ser. Nos. ______ entitled “Method For Setting Tone Controls During a Hearing Test,” ______ entitled “Method For Muting and/or Un-Muting of Audio Sources During a Hearing Test,” and ______ entitled “Method For Setting Volume and/or Balance Controls During a Hearing Test,” filed on even date herein. Each of the above Patent Applications is hereby incorporated by reference.

### FIELD OF THE INVENTION

The present invention generally relates to audiology and the presentation of audio signals for assessing a person's hearing using a personal computer. More specifically, the present invention relates to a system and a method for allowing people to test their hearing at home using conventional, commercially available computer systems.

### BACKGROUND

Audiometric equipment exists for testing hearing. However, such equipment is expensive and is generally available only in hearing clinics. Many people are reluctant to visit hearing clinics and take a hearing test for a variety of reasons. Such reasons may include the cost of a hearing test, the time and inconvenience involved in scheduling of an appointment, waiting for and undergoing a hearing test, and privacy concerns. As a result, as many as 80 percent of the people who suffer from hearing loss in the United States may have not had their hearing tested.

Currently, a number of companies provide hearing tests over the Internet. For example, a user may have his/her hearing tested by accessing one of the following URLs: www.didyouhearme.com, www.handtronix.com, www.onlinehearing.com, www.audiainc.com, www.nigelworks.com or www.freehearingtest.com. For example, when a user accesses www.didyouhearme.com, the website generates a 500 Hz tone and instructs the user to decrease the volume on the user's computer until the user can no longer hear the 500 Hz tone. Next, the website generates a 4000 Hz tone. According to the website, if the user cannot hear the 4000 Hz tone, the user may have a hearing impairment. Next, the website generates a number of tones at different amplitudes at the following frequencies: 250, 500, 750, 1000, 1500, 2000, 3000, and 4000 Hz. When the user hears a particular frequency, the user presses an acknowledge button on the screen with the computer's mouse. Based upon the user's acknowledgements, the website generates an audiogram. An audiogram is a chart plotting a user's hearing threshold level in dB HL as a function of frequency. Using the audiogram, the user can determine if the user has a hearing impairment.

In order to perform the above hearing test, websites present audio represented in the form of sound files stored on the server. In some implementations, these files are downloaded from the server as a particular sound is needed. For example, the audio test on www.didyouhearme.com downloads audio files as part of a Flash/Shockwave package and also plays some sound clips using the java AudioClip applet. In other audio tests, the user is required to download the files in advance. For example, setting up for the audio test offered by www.audiainc.com requires the user to download about 200 audio files onto the user's computer. The audio test is then conducted by selectively playing these audio files. In the case of the www.audiainc.com audio test, the size of each audio file is 10000 bytes. Transferring such a large number of files is not optimal. First, the time required to transfer such recordings can be significant, whether the transfer is implicit like in the Flash/Shockwave implementation or explicit as is the case with www.audiainc.com. Thus, the user may not be able to begin the hearing test until some or all of the recordings have been transferred. Second, the user is only able to play the downloaded recordings. Thus, the user is limited in the number of different frequency/amplitude level stimuli that can be included in each hearing test. As a result, the user cannot customize the hearing test.

Finally, relying on just playing sound files puts a practical limit on the type of signals that can be produced. One example of this is stimulus signals with very long cycle times. Downloading a short audio file and then looping the file cannot generate such signals. These kinds of signals are often utilized in some types of hearing tests.

Thus, improved methods of testing a person's hearing are needed.

### SUMMARY OF THE INVENTION

One embodiment of the invention is a method of testing the hearing of a user utilizing a computer system. The computer system includes a computer and a speaker. The computer can output an electrical signal to the speaker. The speaker can convert the electrical signal into a stimulus. The method includes: downloading a computer program from a server to the computer; executing the computer program on the computer, the execution of the computer program generating an audio stream; based upon the audio stream, generating a stimulus; and receiving an input from the user that indicates that the user heard the stimulus. In some embodiments of the invention, the method also includes: sending data to the server; qualifying the hearing of the user; and sending other data to the computer.

Still other embodiments of the invention include program storage devices that contain computer readable instructions that, when executed, perform portions of the above methods.

### BRIEF DESCRIPTION OF THE FIGURE

FIG. 1 presents a method of testing the hearing of a user that includes generating an audio stream.

### DESCRIPTION OF THE PREFERRED EMBODIMENTS

The following description is presented to enable any person skilled in the art to make and use the invention, and is provided in the context of a particular application and its requirements. Various modifications to the disclosed embodiments will be readily apparent to those skilled in the art, and the general principles defined herein may be applied to other embodiments and applications without departing from the spirit and scope of the present invention. Thus, the present invention is not intended to be limited to the embodiments shown, but is to be accorded the widest scope consistent with the principles and features disclosed herein.

### Hearing Tests that Locally Generate Audio Streams

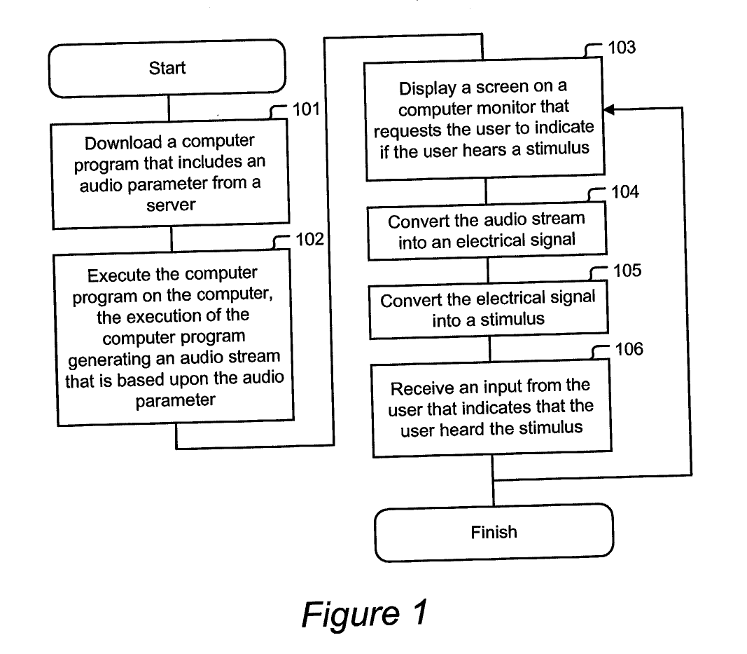

One embodiment of the invention, a method of testing the hearing of a user utilizing a computer system, is shown in FIG. 1. The method can be performed on a conventional computer system, such as a desktop computer system, a laptop computer system, or a handheld computer system. Other devices that include a microprocessor, such as a telephone, a mobile phone, a personal display assistant, an MP3 player, a radio, or a television, can also perform the method. In the method shown in FIG. 1, an audio stream, i.e., digital data that represents an audio signal, is generated by the computer. Thus, the audio streams are not downloaded from a server to a user's computer. By locally generating the audio streams, the download time of the computer program can be significantly reduced, the flexibility of the hearing test can be greatly increased, the complexity of the hearing test can be greatly increased, and the range of signals that can be incorporated in the test can be greatly increased.

Referring to Block 101 of FIG. 1, a user that desires to take a hearing test first downloads a computer program, such as a stand-alone executable program, a Java applet, an Active X control, or a Netscape plugin, from another computer, such as a server, to his/her computer. In one embodiment of the invention, the computer program is transferred via the Internet. In another embodiment of the invention, the computer program is transferred via an email. As is well known, computer programs may be attached to emails that can be easily distributed over the Internet, virtual private networks, local area networks and/or wide area networks. In still other embodiments, the computer program could be transferred to the user via the United States postal service or other postal service.

In some embodiments of the invention, the computer program would include one or more audio parameters. For example, the computer program could include audio parameters that indicate frequencies of stimuli. In one embodiment of the invention, the computer program includes parameters that indicate the following frequencies: 125 Hz, 250 Hz, 500 Hz, 750 Hz, 1khz, 1500 Hz, 2 kHz, 3 kHz, 4 kHz, 6 kHz, and 8 kHz. In some embodiments of the invention, the computer program would also include a number of audio parameters that indicate the amplitudes of stimuli. For example, the computer program may include parameters that indicate the amplitudes such as: +10 dB, +6 dB, +3 dB, 0 dB, −3 db, −6 dB, and −10 dB. In some embodiments of the invention, the computer program would include one or more audio parameter pairs that indicate both the frequency and the amplitude of stimuli. In still other embodiments of the invention, the computer program would include a number of audio parameters that indicate the type of stimulus. For example, the computer program may include parameters that indicate the following stimulus types: Warble tone, white noise, pink noise, shaped noise, high-pass, low-pass, band-pass, and/or sinusoid. In some embodiments of the invention, the computer program may include one or more parameters that indicate that one or more of the above stimulus types should be combined to generate a combined stimulus.

Next, referring to Block 102 of FIG. 1, the user would execute the computer program on the user's computer. When the computer program is executed, the computer program would generate an audio stream based upon one or more parameters that are included in the computer program.

Referring to block 103 of FIG. 1, the computer program would then display a screen on the computer monitor that requests the user to indicate if the user hears a stimulus. Next, referring to Blocks 104 and 105 of FIG. 1, the computer program would convert the audio stream into an electrical signal, which is converted into a stimulus by a speaker, such as a computer speaker, a headphone or any other transducer including a piezo electric transducer. If the user hears the stimulus, then the user would input information into the computer that indicates that the user heard the stimulus. Referring to Block 106 of FIG. 1, the computer program would then receive the input from the user that indicates whether or not the user hears the stimulus.

As shown in FIG. 1, by repeating Blocks 103 through 106 with stimuli of different amplitudes and frequencies, data sufficient to quantify the hearing of the user can be derived using conventional methods. In some embodiments of the invention, the computer program qualifies the hearing. In other embodiments, the computer program transfers data to a server and the server qualifies the hearing and then sends data back to the computer program. After the hearing of the user is quantified, some embodiments of the invention present an audiogram, text information, and/or graphical information to the user.

There are many advantages that result from the computer program generating audio streams. First, the time required to download the computer program can be dramatically reduced if the computer generates the audio streams after the computer program has been downloaded because the audio streams need not be downloaded. Second, because the size of the computer program can be reduced, the load upon the server that provides the computer program to the computer can also be reduced.

Third, because the user is not limited to the audio streams that were downloaded with the computer program, the flexibility and complexity of the hearing test can be increased. For example, a large number of stimuli of varying types, some of which have long cycle times, could be utilized. In fact, in some embodiments of the invention, the user may customize the hearing test. For example, if the user is particularly concerned about the user's hearing in a narrow frequency range, then the computer program could generate a number of stimuli of different types within a user-defined frequency range.

Finally, some hearing tests may incorporate several stages. One stage could be considered a calibration stage where the user must perform certain actions in response to audio stimuli with the purpose to setup the user's computer system for the actual test. Another stage would be the actual test itself. It is conceivable that the signals required during the first stage would be very different from the ones required for the second stage. Having the audio generated internally by the program on the user's computer makes it more feasible to have completely different signal types for the different stages.

### Other Embodiments of the Invention

Numerous methods of testing the user's hearing have been described. In some embodiments of the invention, several of these methods can be combined. For example, a hearing test may include setting a volume control, setting a tone control, muting audio sources, generating audio streams, generating non-probe stimuli that are outside of the audible range of humans and/or generating stimuli that include inaudible sub-stimuli.

In addition, other embodiments of the invention may further reduce background noise by turning off certain computer devices. For example, any of the above methods may include turning off a computer hard disk drive, floppy disk drive, CD drive, and/or DVD drive. Still other embodiments of the invention would request a user to turn off such devices.

In still other embodiments of the invention, the computer could generate audio streams based not only upon parameters received from the computer program and user input. In these embodiments, the audio streams could be generated based in part upon the hardware present in the computer system. For example, the computer program may query the sound card to determine its manufacturer and model number. Alternatively or in addition to, the computer program may request that the user identify the manufacturer and model number of the speakers that are coupled to the computer.

Still other embodiments of the invention include a program storage device such as described in the Summary of the Invention.

### Conclusion

The foregoing descriptions of embodiments of the present invention have been presented for purposes of illustration and description only. They are not intended to be exhaustive or to limit the present invention to the forms disclosed. Accordingly, many modifications and variations will be apparent to practitioners skilled in the art. For example, program storage devices, such as hard disks, floppy disks, random access memories (RAM), read only memories (ROM), programmable read only memories (PROM), compact disks (CD), and digital versatile disks that contain computer readable instructions that perform portions of the above methods, are intended to be included in the present invention. Additionally, the above disclosure is not intended to limit the present invention. The scope of the present invention is defined by the appended claims.

It is claimed:

1. A method of testing the hearing of a user utilizing a computer system, the computer system including a computer and a speaker, the computer operable to output an electrical signal to the speaker, the speaker operable to convert the electrical signal into a stimulus, the method comprising:
a) downloading a computer program from a server to the computer;
b) executing the computer program on the computer, the execution of the computer generating an audio stream;
c) based upon the audio stream, generating a stimulus; and
d) receiving an input from the user that indicates if the user heard the stimulus.

2. The method of claim 1, wherein the computer program includes an audio parameter and wherein the audio stream is generated based upon the audio parameter.

3. The method of claim 1, wherein the act of downloading the computer program includes transferring the computer program from the server to the computer via the Internet.

4. The method of claim 1, wherein the act of downloading the computer program includes transferring the computer program from the server to the computer via an email.

5. The method of claim 1, wherein the act of downloading the program from a server includes downloading an audio parameter that indicates at least one frequency of the stimulus.

6. The method of claim 1, wherein the act of downloading the program from a server includes downloading an audio parameter that indicates at least one amplitude of the stimulus.

7. The method of claim 1, wherein the act of downloading the program from a server includes downloading an audio parameter that indicates at least one type of the stimulus.

8. The method of claim 1, wherein the act of downloading the program from a server includes downloading an audio parameter that indicates that two stimulus types should be combined to generate the stimulus.

9. The method of claim 1, wherein the act of downloading the program from a server includes downloading an audio parameter that indicates that the program should determine which stimulus should be presented in the test.

10. The method of claim 1, wherein the act of generating a stimulus includes generating a stimulus within a user-defined frequency range.

11. The method of claim 1, further including:
a) sending first data to the server;
b) qualifying the hearing of the user; and
c) sending second data to the computer.

12. A method of testing the hearing of a user utilizing a computer system, the computer system including a computer and a speaker, the computer operable to output an electrical signal to the speaker, the speaker operable to convert the electrical signal into a stimulus, the method comprising:
a) downloading a computer program from a server to the computer;
b) executing the computer program on the computer, the execution of the computer program generating an audio stream;
c) based upon the audio stream, generating a stimulus; and
d) receiving an input from the user that indicates that the user heard the stimulus.

13. The method of claim 12, wherein the act of downloading the computer program includes transferring the computer program from the server to the computer via the Internet.

14. The method of claim 12, wherein the act of downloading the computer program includes transferring the computer program from the server to the computer via an email.

15. The method of claim 12, wherein the act of generating a stimulus includes generating a first stimulus having a first frequency and a second stimulus having a second frequency.

16. The method of claim 12, wherein the act of generating a stimulus includes generating a first stimulus having a first amplitude and a second stimulus having a second amplitude.

17. The method of claim 12, wherein the act of generating a stimulus includes generating a first stimulus having a first type and a second stimulus having a second type.

18. The method of claim 12, further including:
a) sending first data to the server;
b) qualifying the hearing of the user; and
c) sending second data to the computer.

19. A program storage device that contains computer readable instructions that, when executed by a computer system, tests the hearing of a user by:
a) generating an audio stream based upon an audio parameter;
b) based upon the audio stream, generating a stimulus;
c) receiving an input from a user that indicates that the user heard the stimulus;
d) sending first data to a server; and
e) receiving second data from the server.

20. The program storage device of claim 19, wherein the audio parameter indicates the frequency of the stimulus.

21. The program storage device of claim 19, wherein the audio parameter indicates the amplitude of the stimulus.

22. The program storage device of claim 19, wherein the audio parameter indicates the type of the stimulus.

23. The program storage device of claim 19, wherein the audio parameter indicates that two stimulus types should be combined to generate the stimulus.

24. The program storage device of claim 19, wherein the act of generating the stimulus includes generating a stimulus within a user-defined frequency range.
